(19) United States
(12) Patent Application Publication
(10) Pub. No.: US 2020/0171268 A1
(43) Pub. Date: Jun. 4, 2020

(54) Heating Weighted Blanket and Accessory

(71) Applicant: Li Zhang, Hangzhou City (CN)

(72) Inventor: Li Zhang, Hangzhou City (CN)

(21) Appl. No.: 16/676,548

(22) Filed: Nov. 7, 2019

(30) Foreign Application Priority Data
Nov. 20, 2018 (CN) 201811383596.2

(51) Int. Cl. A61M 21/02; H05B 3/34; A47G 9/02; H05B 3/14; A61M 21/00

(52) U.S. Cl. CPC A61M 21/02; H05B 3/342; A47G 9/0215; H05B 3/145; A61M 2205/0205; A61M 2205/3646; A61M 2021/0066; A61M 2021/0022; A61M 2210/0612; H05B 2203/013

(57) ABSTRACT

The present invention discloses a heating weighted blanket and accessory, including a blanket and accessory sleeve body, where balance weight areas are evenly distributed in the blanket and accessory sleeve body, balance weight beads are disposed in the balance weight areas, and an inner side of the balance weight area is partially provided with a heating layer. The heating layer is a graphene heating sheet, and the heating layer is connected to a power switch interface on an outer surface of the blanket and accessory sleeve body. Filling balance weights of the present invention are reasonable, the combination of a filler and a blanket and accessory is complete, and the balance weights can be evenly distributed on the blanket and accessory, are not partially overweight and are good in liquidity. Moreover, the blanket and accessory has a rapid heating mode, enables a user to quickly enter a warm state in winter and is high in comfort. Besides, in conjunction with the weighted blanket and accessory, pressure can be released, and anxiety is relieved.

### TECHNICAL FIELD

The present invention relates to the field of bedding, and in particular, to a heating weighted blanket and accessory.

### BACKGROUND

As the pace of life in modern society continuously accelerates, sub-health has become a common problem for modern people. People are getting more and more stressed and their bodies are overloaded, which brings more sleep and emotional problems.

In the field of occupational therapy in the United States, a product called “weighted blanket” has been popular, and the main feature thereof is that the weight of the blanket applied to the human body is more than 10% of the body weight. Investigations have shown that weighted blanket has a general effect of relieving anxiety and relaxing, and can improve the quality of sleep in people with insomnia. Research on the use of weighted blanket to improve a sleep state and reduce stress has been around for some time and has been recognized by users.

Therefore, the study of the blanket and accessory with weighted structures needs further development, and heating in winter is an important issue. How to keep warm quickly is an important issue. In conjunction with the fact that the effects of relieving anxiety, relaxing and alleviating stress can be achieved when the weight applied to the human body should exceed 10% of the body weight, it is imperative to develop a blanket and accessory having functions of quick heating, anxiety relief and relaxation.

### SUMMARY

To solve the foregoing problems, the present invention provides a heating weighted blanket and accessory, filling balance weights thereof are reasonable, the combination of a filler and a blanket and accessory is complete, and the balance weights can be evenly distributed on the blanket and accessory, are not partially overweight and are good in liquidity. Moreover, the blanket and accessory has a rapid heating mode, enables a user to quickly enter a warm state in winter and is high in comfort. Besides, in conjunction with the weighted blanket and accessory, pressure can be released, and anxiety is relieved.

Moreover, the blanket and accessory has a variety of structural realization forms and is highly practical.

To achieve the foregoing objective, the technical solution adopted by the present invention includes a blanket and accessory sleeve body, where balance weight areas are evenly distributed in the blanket and accessory sleeve body, balance weight beads are disposed in the balance weight areas, and an inner side of the balance weight area is partially provided with a heating layer. The heating layer is a graphene heating sheet, and the heating layer is connected to a power switch interface on an outer surface of the blanket and accessory sleeve body.

Further, the blanket and accessory sleeve body is an eye patch sleeve body including an eye patch body and elastic band bodies connected to both ends of the eye patch body, and the eye patch body is divided into two left and right balance weight areas by a longitudinal quilting seam.

Further, the heating layers are disposed at the bottom of the left and right balance weight areas, and the power switch interface is located at a top side edge position.

Further, polyester wadding is disposed between a lower side of the balance weight bead and the eye patch body, and the heating layer is clamped therein.

In addition, the blanket and accessory sleeve body can also be a U-shaped pillow.

Specifically, the U-shaped pillow includes a U-shaped pillow cover body, and the balance weight areas are detachably disposed in the U-shaped pillow cover body through an inner core sleeve body.

Further, the heating layer is disposed on bottom sides of adjacent balance weight areas at an arc top of the inner core sleeve body, and the power switch interface is detachably located on an outer side of an arc top edge through the U-shaped pillow cover body.

Further, polyester wadding is disposed between a lower side of the balance weight bead and the inner core sleeve body, and the heating layer is clamped therein.

Further, the blanket and accessory sleeve body may also be a shawl including a shawl body, and the balance weight area is formed by transverse and longitudinal quilting seams.

Further, the heating layer is disposed on bottom sides of the balance weight areas at neck and shoulder positions of the shawl body, and the power switch interface is located on a downward extension area of the shawl body.

Further, polyester wadding is disposed between a lower side of the balance weight bead and the shawl body, and the heating layer is clamped therein.

Moreover, the blanket and accessory sleeve body may also be a blanket including a blanket body, and the balance weight area is formed by transverse and longitudinal quilting seams.

Further, the heating layer is disposed on bottom sides of a plurality of adjacent balance weight area at a middle portion of the blanket body, and the power switch interface is located at an edge position of the blanket body.

Further, polyester wadding is disposed between a lower side of the balance weight bead and the blanket body, and the heating layer is clamped therein.

In the foregoing blanket and accessory, an upper surface of the blanket and accessory sleeve body is a heat insulation layer, and a lower surface thereof is a far-infrared antibacterial blanket and accessory layer.

Further, the balance weight beads in the single balance weight area are 60-100 g, and the balance weight beads are each a silicon dioxide glass bead having a diameter of 0.8-1.6 mm

Further, the polyester wadding has a square gram weight of 100-300 g.

In summary, the present invention has the following advantages:

The present invention stimulates pressure points of the human body with appropriate mass to improve emotions and relax the body and mind. At the same time, the weighting is achieved in a mode of filling of the weighted blanket and accessory, and the weighted blanket and accessory has a heating function.

According to the present invention, the balance weight areas are divided by the quilting seams, so that the filler and a blanket body are well combined. The filler is evenly distributed on the product, and the product is kept soft, is foldable and keeps warm. The covering experience is good, and uniform distribution of the weight is achieved. The balance weight beads are evenly distributed on the whole blanket, so that the partial weight is not too heavy; and the balance weight beads have certain fluidity in the balance weight area, so that the overall comfort of the blanket is improved.

In addition, a heating sheet adopts the graphene heating sheet, which can perform partial heating for 5 s, thereby improving the user's comfort. Moreover, the blanket and accessory has various structural realization forms, is highly practical, and uses various environments.

### BRIEF DESCRIPTION OF THE DRAWINGS

FIG. 1 is a structural reference diagram of the present invention;

FIG. 2 is a schematic diagram of an embodiment;

FIG. 3 is a schematic diagram of a preferred embodiment;

FIG. 4 is a schematic diagram of another embodiment; and

FIG. 5 is a schematic diagram of a further embodiment.

### DESCRIPTION OF THE EMBODIMENTS

The present invention will be further described below with reference to the accompanying drawings and embodiments.

### Embodiment 1

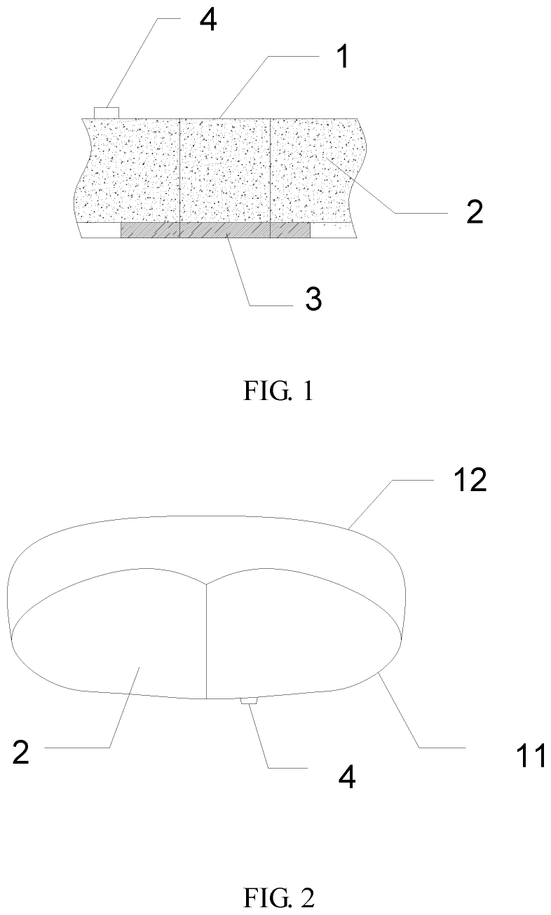

A heating weighted blanket and accessory, as shown in FIGS. 1 and 2, includes a blanket and accessory sleeve body 1, where balance weight areas 2 are evenly distributed in the blanket and accessory sleeve body 1, balance weight beads are disposed in the balance weight areas 2, and an inner side of the balance weight area is partially provided with a heating layer 3. Specifically, the heating layer 3 is a graphene heating sheet which is preferably a graphene heating sheet from Wuxi Gefei Electronic Film Technology Co., Ltd., or a graphene heating sheet from Shenzhen Haideyide Technology Co., Ltd. The graphene heating sheet can perform partial heating for 5 s, thereby improving the user's comfort.

The heating layer 3 is connected to a power switch interface 4 on an outer surface of the blanket and accessory sleeve body 1, thereby enabling a user to independently select whether to use a heating function.

Specifically, referring to FIG. 2, the blanket and accessory sleeve body 1 is an eye patch sleeve body specifically including an eye patch body 11 and elastic band bodies 12 connected to both ends of the eye patch body 11.

The eye patch body 11 is divided into two left and right balance weight areas 2 by a longitudinal quilting seam, the balance weight beads in the single balance weight area 2 are 60-100 g, and the balance weight beads are each a silicon dioxide glass bead having a diameter of 0.8-1.6 mm

An upper surface of the eye patch body 11 is a heat insulation layer, and a lower surface thereof is a far-infrared antibacterial blanket and accessory layer. The heat insulation layer is preferably a flannel layer or a polar fleece layer, and the far-infrared antibacterial blanket and accessory layer is preferably a far-infrared cloth from Changshu Dongfang Warp & Weft Knitting Co., Ltd.

In specific arrangement, the heating layers are disposed at the bottom of the left and right balance weight areas 2, and the power switch interface 4 is located at a top side edge position.

To improve the use comfort of the blanket and accessory, polyester wadding is disposed between a lower side of the balance weight bead and the eye patch body 11, and the heating layer 3 is clamped therein. The polyester wadding has a square gram weight of 100-300 g, preferably 100 g in this embodiment.

### Embodiment 2

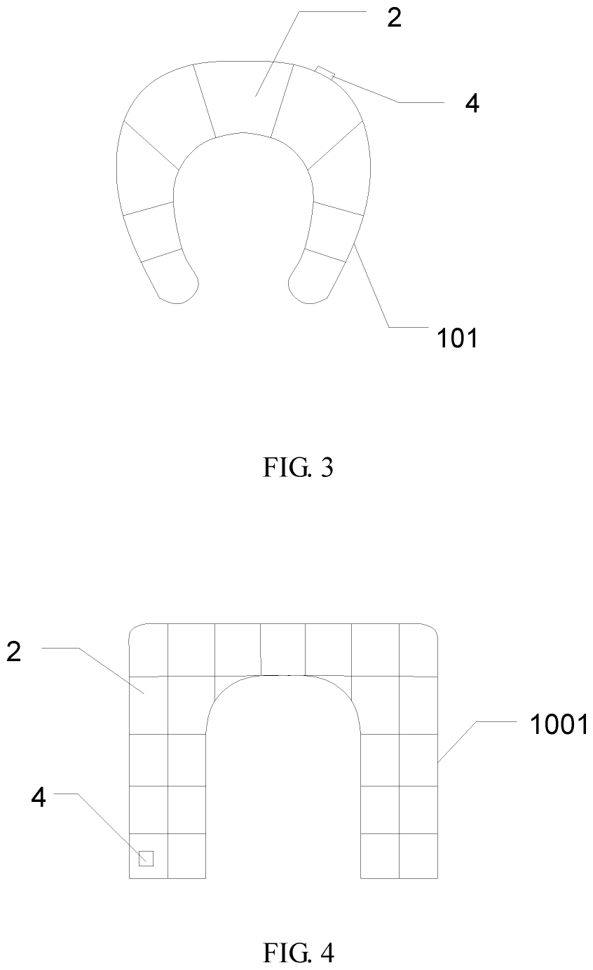

Referring to FIG. 3, the blanket and accessory sleeve body 1 is a U-shaped pillow.

Specifically, the U-shaped pillow includes a U-shaped pillow cover body 101, an upper surface of the eye patch body 101 is a heat insulation layer, and a lower surface thereof is a far-infrared antibacterial blanket and accessory layer. The heat insulation layer is preferably a flannel layer or a polar fleece layer, and the far-infrared antibacterial blanket and accessory layer is preferably a far-infrared cloth from Changshu Dongfang Warp & Weft Knitting Co., Ltd.

The balance weight areas 2 are detachably disposed in the U-shaped pillow cover body 101 through an inner core sleeve body 5, which can facilitate cleaning. The balance weight beads in the single balance weight area 2 are 60-100 g, preferably 60 g in this embodiment.

The balance weight beads each adopt a silicon dioxide glass bead having a diameter of 0.8-1.6 mm, preferably 0.8 mm in this embodiment.

The heating layer 3 is specifically a graphene heating layer and is disposed on bottom sides of adjacent balance weight areas 2 at an arc top of the inner core sleeve body 5, and the power switch interface 4 is detachably located on an outer side of an arc top edge through the U-shaped pillow cover body 101. During use, the neck and shoulder positions of the user are heated, and the comfort is improved.

To improve the use comfort, polyester wadding is disposed between a lower side of the balance weight bead and the inner core sleeve body 5, and the heating layer 3 is clamped therein. The polyester wadding has a square gram weight of 100-300 g, preferably 300 g in this embodiment.

### Embodiment 3

Referring to FIG. 4, the blanket and accessory sleeve body 1 is a shawl.

Specifically, the shawl includes a shawl body 1001. An upper surface of the shawl body is a heat insulation layer, and a lower surface thereof is a far-infrared antibacterial blanket and accessory layer. The heat insulation layer is preferably a flannel layer or a polar fleece layer, and the far-infrared antibacterial blanket and accessory layer is preferably a coconut carbon far-infrared cloth from Changshu Dongfang Warp & Weft Knitting Co., Ltd.

The balance weight area 2 is formed by transverse and longitudinal quilting seams. The balance weight beads in the single balance weight area 2 are 60-100 g, preferably 80 g in this embodiment.

The balance weight beads each adopt a silicon dioxide glass bead having a diameter of 0.8-1.6 mm, preferably 1.6 mm in this embodiment.

The heating layer 3 is disposed on bottom sides of the balance weight areas 2 at neck and shoulder positions of the shawl body 1001, to achieve partial heating of the neck and shoulder parts.

The power switch interface 4 is located on a downward extension area of the shawl body 1001, which is convenient for the user to switch freely.

To improve the use comfort, polyester wadding is disposed between a lower side of the balance weight bead and the shawl body 1001, and the heating layer 3 is clamped therein. The polyester wadding has a square gram weight of 100-300 g, preferably 180 g in this embodiment.

### Embodiment 4

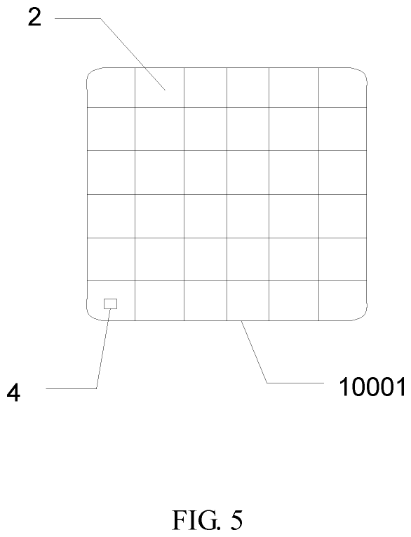

Referring to FIG. 5 the blanket and accessory sleeve body 1 is a blanket.

Specifically, the blanket includes a blanket body 10001. An upper surface of the blanket body is a heat insulation layer, and a lower surface thereof is a far-infrared antibacterial blanket and accessory layer. The heat insulation layer is preferably a flannel layer or a polar fleece layer, and the far-infrared antibacterial blanket and accessory layer is preferably a far-infrared cloth from Changshu Dongfang Warp & Weft Knitting Co., Ltd.

The balance weight area 2 is formed by transverse and longitudinal quilting seams. The balance weight beads in the single balance weight area 2 are 60-100 g, preferably 100 g in this embodiment.

The balance weight beads each adopt a silicon dioxide glass bead having a diameter of 0.8-1.6 mm, preferably 1.1 mm in this embodiment.

The heating layer 3 is disposed on bottom sides of a plurality of adjacent balance weight area 2 at a middle portion of the blanket body 10001, and the power switch interface 4 is located at an edge position of the blanket body 10001.

To improve the use comfort, polyester wadding is disposed between a lower side of the balance weight bead and the blanket body 10001, and the heating layer 3 is clamped therein. The polyester wadding has a square gram weight of 100-300 g, preferably 200 g in this embodiment.

The foregoing embodiments are merely illustrative of the technical concept and features of the present invention, the purpose is to enable those skilled in the art to understand the content of the present invention and to implement the present invention, and this does not limit the protection scope of the present invention. Equivalent transformations or modifications made in accordance with the spirit of the present invention should fall within the protection scope of the present invention.

1. A heating weighted blanket and accessory, comprising a blanket and accessory sleeve body (1), wherein balance weight areas (2) are evenly distributed in the blanket and accessory sleeve body (1), balance weight beads are disposed in the balance weight areas (2), and an inner side of the balance weight area is partially provided with a heating layer (3); the heating layer (3) is a graphene heating sheet, and the heating layer (3) is connected to a power switch interface (4) on an outer surface of the blanket and accessory sleeve body (1).

2. The heating weighted blanket and accessory according to claim 1, wherein the blanket and accessory sleeve body (1) is an eye patch sleeve body comprising an eye patch body (11) and elastic band bodies (12) connected to both ends of the eye patch body (11), and the eye patch body (11) is divided into two left and right balance weight areas (2) by a longitudinal quilting seam.

3. The heating weighted blanket and accessory according to claim 2, wherein the heating layers (3) are disposed at the bottom of the left and right balance weight areas (2), and the power switch interface (4) is located at a top side edge position.

4. The heating weighted blanket and accessory according to claim 2, wherein polyester wadding is disposed between a lower side of the balance weight bead and the eye patch body (11), and the heating layer (3) is clamped therein.

5. The heating weighted blanket and accessory according to claim 1, wherein the blanket and accessory sleeve body (1) is a U-shaped pillow comprising a U-shaped pillow cover body (101), and the balance weight areas (2) are detachably disposed in the U-shaped pillow cover body (101) through an inner core sleeve body (5).

6. The heating weighted blanket and accessory according to claim 5, wherein the heating layer (3) is disposed on bottom sides of adjacent balance weight areas (2) at an arc top of the inner core sleeve body (5), and the power switch interface (4) is detachably located on an outer side of an arc top edge through the U-shaped pillow cover body (101).

7. The heating weighted blanket and accessory according to claim 5, wherein polyester wadding is disposed between a lower side of the balance weight bead and the inner core sleeve body (5), and the heating layer (3) is clamped therein.

8. The heating weighted blanket and accessory according to claim 1, wherein the blanket and accessory sleeve body (1) is a shawl comprising a shawl body (1001), and the balance weight area (2) is formed by transverse and longitudinal quilting seams.

9. The heating weighted blanket and accessory according to claim 8, wherein the heating layer (3) is disposed on bottom sides of the balance weight areas (2) at neck and shoulder positions of the shawl body (1001), and the power switch interface (4) is located on a downward extension area of the shawl body (1001).

10. The heating weighted blanket and accessory according to claim 8, wherein polyester wadding is disposed between a lower side of the balance weight bead and the shawl body (1001), and the heating layer (3) is clamped therein.

11. The heating weighted blanket and accessory according to claim 1, wherein the blanket and accessory sleeve body (1) is a blanket comprising a blanket body (10001), and the balance weight area (2) is formed by transverse and longitudinal quilting seams.

12. The heating weighted blanket and accessory according to claim 11, wherein the heating layer (3) is disposed on bottom sides of a plurality of adjacent balance weight area (2) at a middle portion of the blanket body (10001), and the power switch interface (4) is located at an edge position of the blanket body (10001).

13. The heating weighted blanket and accessory according to claim 11, wherein polyester wadding is disposed between a lower side of the balance weight bead and the blanket body (10001), and the heating layer (3) is clamped therein.

14. The heating weighted blanket and accessory according to claim 1, wherein an upper surface of the blanket and accessory sleeve body (1) is a heat insulation layer, and a lower surface thereof is a far-infrared antibacterial and deodorant blanket and accessory layer made on the basis of carbon raw materials such as coconut carbon and activated carbon.

15. The heating weighted blanket and accessory according to claim 1, wherein the balance weight beads in the single balance weight area (2) are 60-100 g, and the balance weight beads are each a silicon dioxide glass bead having a diameter of 0.8-1.6 mm.

16. The heating weighted blanket and accessory according to claim 4, wherein the polyester wadding has a square gram weight of 100-300 g.
